(19) United States
(12) Patent Application Publication
(10) Pub. No.: US 2024/0016525 A1
(43) Pub. Date: Jan. 18, 2024

(54) CONTOURED HOOK COMPRESSION PLATE WITH SHEAR-OFF TERMINAL SCREWS FOR FRACTURE FIXATION AND A METHOD OF FIXATION OF A FRACTURE OF THE MEDIAL MALLEOLUS

(71) Applicant: ORTHO SOLUTIONS HOLDINGS LIMITED, Lakewood, CO (US)

(72) Inventors: Brian HOCKETT, Strongsville, OH (US); Andrew LEITHER, Akron, OH (US); Dustin DUCHARME, Littleton, CO (US)

(21) Appl. No.: 18/349,559

(22) Filed: Jul. 10, 2023

Related U.S. Application Data
(60) Provisional application No. 63/388,393, filed on Jul. 12, 2022.

(51) Int. Cl. A61B 17/80

(52) U.S. Cl. CPC A61B 17/8057; A61B 17/8061; A61B 2017/00004

(57) ABSTRACT

A bone fixation plate for fixation of the medial malleolus and similar bones. The bone fixation plate includes an elongated body portion, held to the long bone by variable locking screws and a curved head that forms a 90° arc to wrap the end of the bone and has a rounded recess to form two prong members that each receive a snap-off screw.

### FIELD OF THE INVENTION

The present invention relates to a contoured orthopedic plate for the fixation of bone fractures, with a particular application to fractures of the medial malleolus. The distal portion of the plate includes a curved hook with two prongs intended to wrap the distal portion of the tibia and accept shear-off screws for fixation over the fracture line.

### BACKGROUND OF THE INVENTION

The prior art has advanced in the innovation and use of orthopedic plates and methods of fixation to include plates contoured for specific anatomic areas and typical fracture patterns. This includes long plates, which may be used on the bones of the lower leg, and to fix typical ankle fractures.

One such example is set forth in U.S. Pat. No. 9,631,157 to Medoff et al. which illustrates a contoured plate that is fixed along an outside edge of the tibia, and which includes spiked hooks that wrap and fix the distal prominence of the bone in the event of a fracture. A problem with the plates shown in this reference is that the spikes are fixed with respect to their length and angle, which can lead to problems in achieving fixation or in a proper anchoring of a distal fragment. In addition, the static nature of the location and angle of the hooks leads to problems with fit such that when the hooks are optimally located, the plate contouring is off so that the plate rocks or the hooks are too steeply angled and don't accommodate the distal portion of the corresponding bone segment. Attempts to customize these spikes intraoperatively adds time and complexities to the surgery, which are not welcome.

Other previous solutions have provided complications even more cumbersome and requiring more cutting or customization of off the shelf hardware.

### OBJECTS AND BRIEF SUMMARY OF THE INVENTION

Accordingly, it is an object of the present invention to provide a bone plate that allows for some pre-operative or intraoperative selection, including for example, selecting a screw length and angle, or allowing a surgeon the ability to tailor the dual hook end by bending one or both hooks legs into a greater angle. The terminal hook screws are typically short sharp spike-like screws that act to fix the terminal fragment and hold it in position while it is buttressed by the wrapping portion of the plate. However, it is possible for a surgeon to select a screw length and angle so that they can cross a fracture line if they determine it to be advantageous. This is not possible with the present plates. Moreover, the flexibility of the screw angle and length, and potential ease of customization of the hook leg ends means that a surgeon can let the plate contouring determine the fit, and the screw characteristics can be easily selected to optimize the fixation of the fragment.

Advantageously, the plate further includes a drill guide which mounts from one of the more proximal holes of the plate to overlay the terminal portion of one quarter to three quarters, and more specifically, one third of the length of the plate with a terminal end having guide bores to direct the insertion of the distal shear off or snap off screws which act as prongs in the associated bone. These screws are selected to secure the distal fragment, and along with an optional compression slot, can be used to create compression across a fracture line, such as in the terminal prominence at or around the medial pyramidal process of the tibia. The screws are designed with an extension for holding the screw prior to implantation and to permit the shearing or snapping of the screw from the extension after implantation. The screws also preferably include an anti-backout feature, such as a flange that interacts with the snap-off screw hole surround to inhibit the screw from backing out over time. In addition, preferably, these screws are self-tapping so that they can be inserted without any additional preparation, such as drilling pilot holes.

It is a further object of the present invention to provide a bone plate that can be seated adjacent against a bone as a result of generalized contouring that provides an accommodating interior (i.e., bone facing) surface which includes a gently radiused end portion to resemble the shape of a J inside profile, and a having a terminal dual pronged end which resembles an inverted U. The prongs include fixation holes for locking or non-locking screws which are preferably variable angle screws, that are preferably snap-off or shear-off screws such that the screws have no or relatively little area which protrudes above the surface of the plate prongs around these fixation holes. The plate is further contoured in outline to minimize the material while allowing sufficient material around the fixation holes to provide the necessary plate and plate/screw interface strength.

Further, it is an object to provide a bone plate that includes a long portion having fixation holes that can accommodate locking or non-locking fixation means, such as screws, and preferable variable angle screws. Ideally, the long portion of the plate includes 2 to 8, and preferably 3 to 5 such holes, wherein one of more of the holes may include compression means, such as a compression slot, shown here in the proximal hole.

The present invention comprises a contoured orthopedic plate for fixing bone fractures having a small terminal fragment. The bone plate has a long bone portion or body having a first end, for example, at the proximal end of the plate, a second end, for example at the distal end of the plate, an exterior or top surface which faces away from the bone in use, an interior or bottom surface which faces toward or is adjacent the bone in use, and serpentine side edges which join the exterior and interior surfaces along the long side, a rounded or circular end edge at the proximal end of the plate and a inwardly curved hook that splits into two or more prongs at an angle of 70° to 110°, and preferably 90°+/−5° to the angle of the long plate long axis. The invention also comprises other contours and specific configurations with respect to the terminal curve and contouring and number of fixation holes, as well as the included compression slot.

The present invention also includes a multiple barreled drill guide facilitating the insertion of at least two parallel terminal screws at the distal end of a bone at the correct location. The multiple barreled drill guide has a drill body, that fastens to the plate, such as by a thumb screw device which is secured in a dedicated threaded hole in the plate and which has outrigger slots for k-wires for helping to temporarily hold the plate in place during screw insertion.

### BRIEF DESCRIPTION OF THE DRAWINGS

FIG. 1 is an Anterior/Posterior (AP) view of a human right distal tibia showing the hook plate and screw system of the present invention in position following surgery;

FIG. 2 is a front view of the hook plate and screw system of FIG. 1;

FIG. 3 is an end view of the hook plate of FIG. 1;

FIG. 4 is a top side view of the hook plate of FIG. 1;

FIG. 5 is a first side view of the hook plate of FIG. 1;

FIG. 6 is a top view of the hook plate of FIG. 1;

FIG. 7 is a second side view of the hook plate of FIG. 1;

FIG. 8 is a top side view of the hook plate of FIG. 1 with fixation screws in position in the plate;

FIG. 9 is a first side view of the hook plate and screw system of FIG. 8;

FIG. 10 is a top view of the hook plate and screw system of FIG. 8;

FIG. 11 is a second top side view of the hook plate and screw system of FIG. 8;

FIG. 12 is a bottom view of the hook plate and screw system of FIG. 8;

FIG. 13 is a medial side view of the plate and drill guide on a distal tibia;

FIG. 14 is an angled side view of the plate and drill guide on a distal tibia as shown in FIG. 13;

FIG. 15 is a side view of the plate and drill guide shown in FIG. 13;

FIG. 16 is a side view of a snap/twist-off screw as used in the present invention;

FIG. 17 is a top side view of the snap/twist-off screw of FIG. 16; and

FIG. 18 is an end view of the snap/twist off screw of FIG. 16.

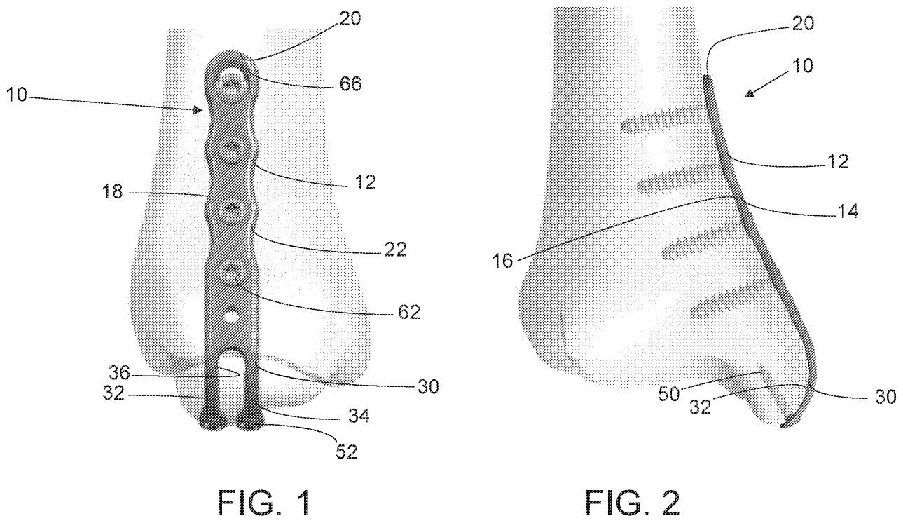

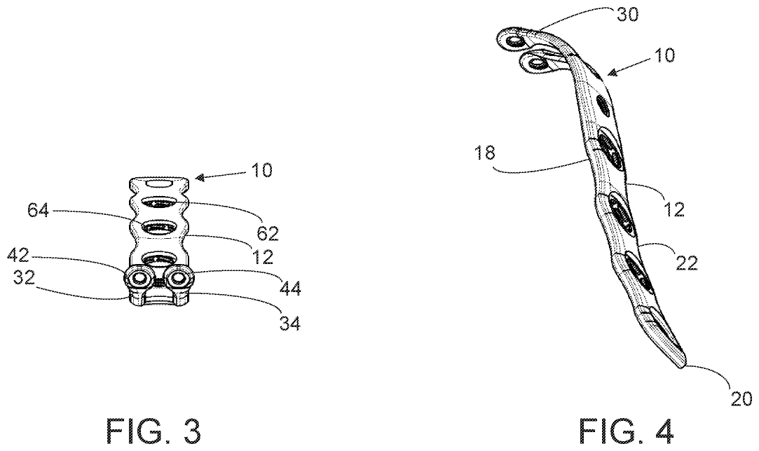

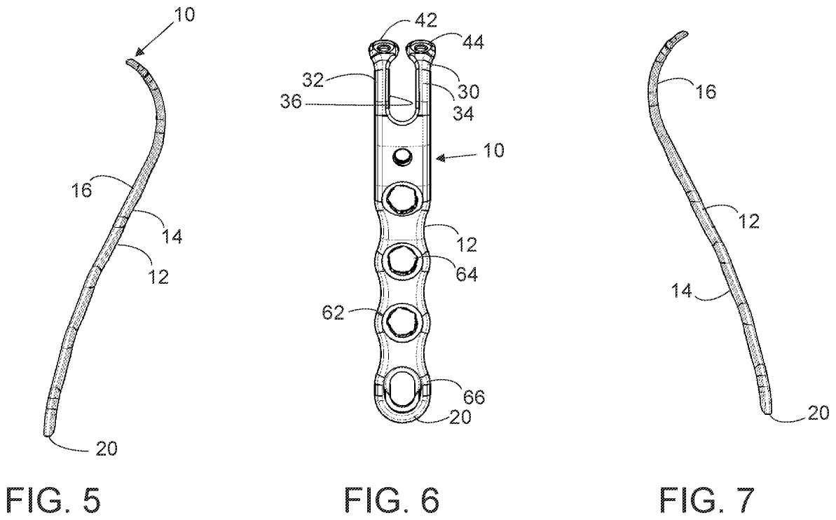

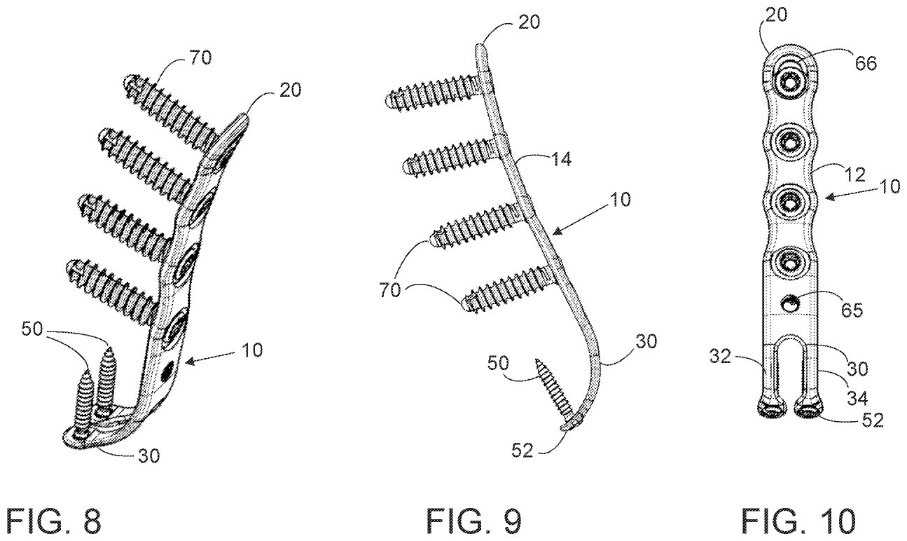

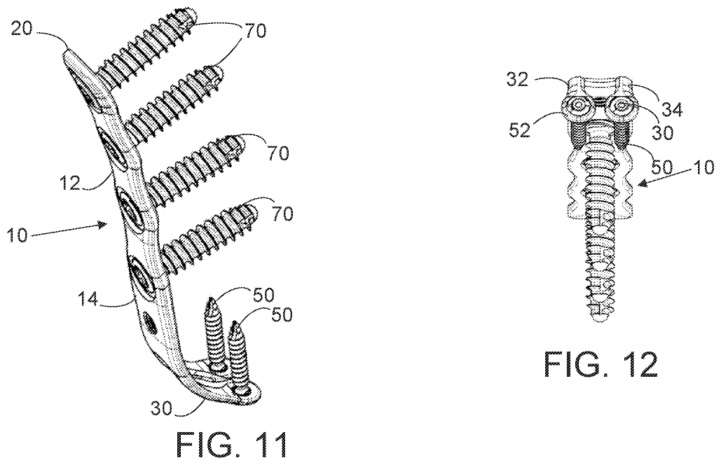

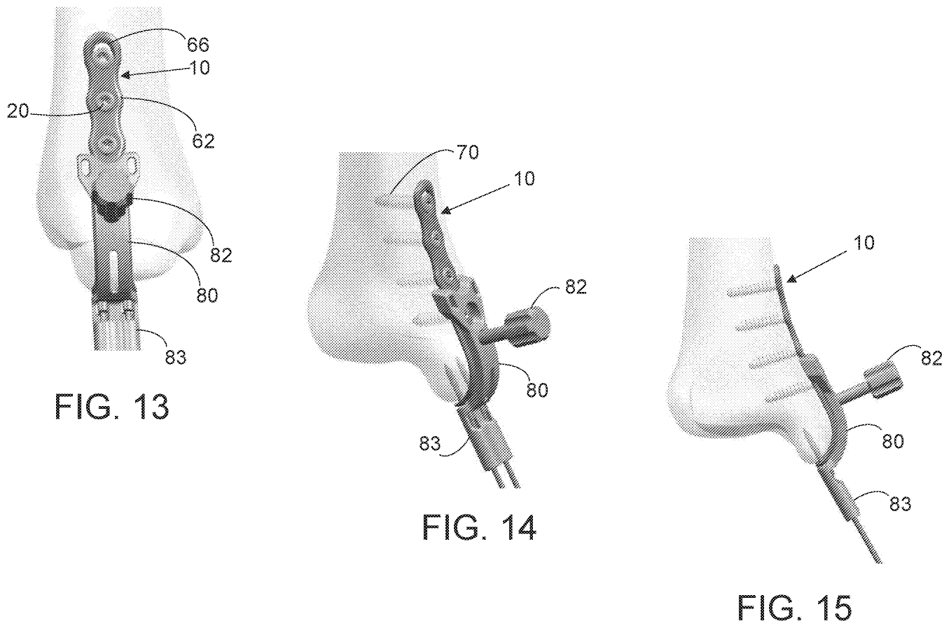

### DETAILED DESCRIPTION OF THE INVENTION

The present invention relates to an orthopedic plate and fastener system to aid in the healing of a bone fracture, in particular, at a terminal end of the bone. The plate 10 has a long body portion 12 which extends along a central axis. This body portion 12 includes an exterior surface 14 which faces away from the bone in use, and an opposing interior surface 16 which faces toward the bone in use. These opposing surfaces are joined by a first side edge 18 on a first side and a spaced apart second edge 22 on a second side. One end of the body portion 12 includes a rounded terminal end 20. The opposing end of the body portion 12 is joined to a curved hook end or head 30 which flows from the central axis in a curve which forms an arc of from 60° to 120°, and more preferably from 70° to 105°, or preferably 90°+/−5°. The head 30 comprises at least two prongs 32, 34 which are separated by a u-shaped recess 36 which extends for at least 75%, and preferably about 90% of the arc and is rounded at the apex to avoid stress concentrations. The screw holes in the prongs could be sized to fit a 3-5 mm screw which is advantageously sharp and self-tapping, however, it is also possible to place a lag screw or cannulated lag screw through this slot/space, so it could be good to at least include that it could accept a lag screw.

The prongs 32, 34 each include a through hole 42, 44 which receive fixation means, and preferably screws 50 which are designed to snap or shear at or below the level of the prong exterior surface 52 surrounding the through holes such that the screw remains captured in the through holes but does not provide a sharp edge to interfere with soft tissue exterior to the implant.

The body portion 12 of the plate 10 also includes through holes 62 for fixation means, and preferably screws 70. These through holes 62 advantageously have an interface with the screws 70 which permit variable angle locking, meaning that the screws can be implanted at a conically varying angle of axis with respect to the center axis of the hole, but subsequently locked into position at the selected angle. In this case, the holes have interrupted internal threads 68 to provide for this feature, as they interact with external threads (not shown) on the body portion screws 70.

The plate of the present invention is specifically designed for use with a medial malleolus and is illustrated on this bone in the drawings. The body portion includes 3 regular screw holes 62, although it is understood, that the plate can be provided in varying lengths and with varying numbers of body screw holes 62 preferably from 2 to 8, or 3+/−1, and one or more compression slot 66, which is a terminal through hole in this case. The body portion includes thickened areas around the body screw holes and thinner areas between the screw holes so as to minimize the total plate material and yet provide sufficient strength for the screw holes 62.

The system also includes a drill guide 80 which is configured to overlay the exterior surface 14 of the plate 10, and which is secured to the plate by a thumb screw member 82 which threads into a threaded hole 65 in the plate included for this purpose. This end of the drill guide includes a double barreled channel member which can be used to drill pilot holes for the terminal screws, or preferably to guide them directly as they are inserted through the holes 42, 44 in the prongs 32, 34 of the plate head.

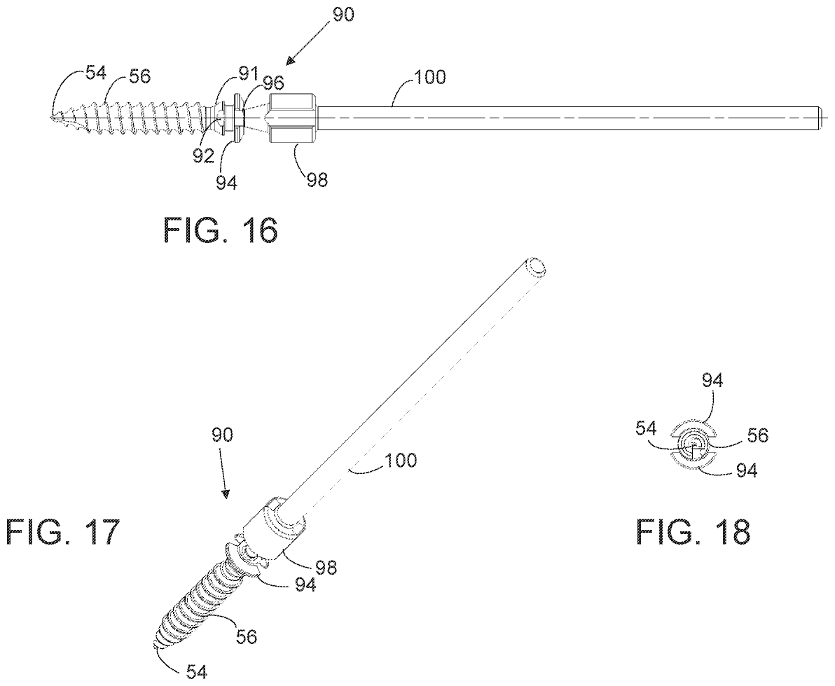

The snap or twist off screws 50 are illustrated in FIGS. 16 and 17 prior to implantation. They include a sharply pointed distal end 54, an external thread 56 and a compound head member 90. The head member 90 has a flared area 91 that includes interruptions 92 and a flange 94 which can be locked into the screw hole to inhibit back-out with a distally facing planar side and moving proximally from there, a necked snap off joint which joins a block 98 and a handle 100. The interruptions help to allow the flange 94 to engage the screw hole internal surfaces.

In use, after exposure of the necessary bone area, the fracture site is cleared and refreshed. Fracture reduction is performed and temporary stabilization is achieved using a K-wire, reduction forceps or a Lobster Claw clamp per the surgeon preference. A drill guide can be used with a hook plate template to locate pilot holes for the plate body screws. After removing the drill guide, the hook plate can be placed over the K-wires and the hook plate can be secured to the bone. A drill guide can be fed over k-wires which extend through the terminal snap off screw holes. The k-wires can be removed and the snap off screws can be implanted into the terminal bone portion. The screws can be snapped off and the flange around the screw head is popped into place in the screw hole to inhibit backing out.

In an additional aspect of the invention the snap off screws are made from a bioabsorbable material. Examples of suitable materials include hydroxyapatite, polylactides, polyglycosides, polydioxanes, magnesium, silicon nitride and human bone.

In accordance with the patent statutes, the best mode and preferred embodiment have been set forth; the scope of the invention is not limited thereto, but rather by the scope of the attached claims.

What is claimed is:

1. A bone plate system comprising:
at least two fixation members and
a bone plate comprising an body portion having a first end, a second end, an exterior surface, and an interior surface and defining body portion through holes between the exterior surface which receive body portion fixation members and the interior surface and one end of the body portion being joined to a head which forms at least 45° of an arc and includes at least two prong members which define prong through holes that receive prong member fixation members.

2. A bone plate system as set forth in claim 1, wherein the prong member fixation members are snap off screws.

3. A bone plate system as set forth in claim 2, wherein the body portion fixation members are variable angle screws.

4. A bone plate system as set forth in claim 3, wherein the variable angle screws are locking screws.

5. A bone plate system as set forth in claim 4, wherein there are from 2 to 6 body portion through holes.

6. A bone plate system as set forth in claim 2, wherein the head forms at least 60 of an arc.

7. A bone plate system as set forth in claim 6, wherein the head forms at least 90 of an arc.

8. A bone plate system further comprising a drill guide.

9. A bone plate system as set forth in claim 2, wherein the snap off screws have a necked area designed to snap-off below the exterior surface of the plate and an anti-backout feature.

10. A bone plate system comprising:
at least two fixation members and
a bone plate comprising n body portion having a first end, a second end, an exterior surface, and an interior surface and defining from 2 to 6 body portion screw holes between the exterior surface and the interior surface which receive variable angle screws, and one of the body portion screw holes comprising a compression slot screw holes and one end of the body portion being joined to a head which forms between 60° and 105° of an arc and includes two prong members separated by a recess having a rounded apex and which prong members define prong through holes that receive fixation members that comprise variable angle snap-off screws.

11. A bone plate system as set forth in claim 10, wherein the body portion has from 3 to screw holes.

12. A bone plate system as set forth in claim 11, further including a drill guide.

13. A bone plate system comprising:
at least two fixation members and
a bone plate comprising a body portion that includes fixation member through holes, at least one of which receives a fixation member and wherein at least one fixation member is a snap off screw.

14. A bone plate system as set forth in claim 13, wherein the snap off screw includes an anti-back out mechanism.

15. A bone plate system as set forth in claim 14, wherein the anti-back out mechanism comprises an annular flange that interacts with an edge of the fixation member screw hole.

16. A bone plate system as set forth in claim 15, wherein the annular flange includes a cut-out to enable the flange to flex so as to permit it to engage the edge of the fixation member screw hole.

17. A bone plate system as set forth in claim 2, wherein said snap off screws comprise a bioabsorbable material.

18. A bone plate system as set forth in claim 11, wherein said snap off screws comprise a bio absorbable material.

19. A bone plate system as set forth in claim 13, wherein said snap off screws comprise a bio absorbable material.
